(19) United States
(12) Patent Application Publication
(10) Pub. No.: US 2026/0083807 A1
(43) Pub. Date: Mar. 26, 2026

(54) INHALANT DEVICE

(71) Applicant: The Trustees of Columbia University in the City of New York, New York, NY (US)

(72) Inventor: Michael Pitman, New York, NY (US)

(21) Appl. No.: 19/408,751

(22) Filed: Dec. 4, 2025

Related U.S. Application Data
(63) Continuation of application No. PCT/US2024/032617, filed on Jun. 5, 2024.
(60) Provisional application No. 63/506,281, filed on Jun. 5, 2023.
(60) Provisional application No. 63/506,930, filed on Jun. 8, 2023.
(60) Provisional application No. 63/530,163, filed on Aug. 1, 2023.

(51) Int. Cl. A61K 36/185; A61K 9/08; A61K 35/644; A61K 47/02; A61K 47/10; A61K 47/18

(52) U.S. Cl. CPC A61K 36/577; A61K 9/08; A61K 35/644; A61K 47/02; A61K 47/10; A61K 47/183

(57) ABSTRACT

Provided is a formulation useful for treating sore throat, throaty cough, xerostomia, pharyngitis, laryngitis, postoperative pain, dysphagia, laryngeal granuloma, vocal optimization, laryngopharyngeal reflux or dysbiosis, wherein the formulation comprises: an aqueous solution comprising one or more active ingredients selected from the group consisting of marshmallow root, slippery elm extract, aloe vera extract, manuka honey, honey, holy basil, menthol, spearmint, capsaicin, propolis, curcumin, xylitol, turmeric, probiotics, and calendula, the aqueous solution being present as aerosolized particles in a range of 10 to 35 microns.

### CROSS REFERENCE TO RELATED APPLICATIONS

The present application is a continuation of international patent application no. PCT/US2024/032617, “Inhalant Device” (filed Jun. 5, 2024); which application claims priority to and the benefit of U.S. patent application No. 63/506,281, “Inhalant Device” (filed Jun. 5, 2023), U.S. patent application No. 63/506,930, “Inhalant Device” (filed Jun. 8, 2023, and U.S. patent application No. 63/530,163, “Oral Nebulized Drug Parameters For Maximizing Drug Delivery To The Anatomically Normal Larynx” (filed Aug. 1, 2023). All foregoing applications are incorporated herein by reference in their entireties for any and all purposes.

### TECHNICAL FIELD

The present disclosure relates to devices, formulations and methods for the treatment of pharyngitis, laryngitis, tracheitis, and other throat discomfort.

### BACKGROUND

Over 11.7 million patients were seen in the ambulatory setting in 2001 with the diagnosis of pharyngitis, which in most cases mean throat discomfort and inflammation. As most sore throats are self-limited, the annual occurrence of sore throats is much higher than estimated by this impressive number of ambulatory visits alone, and the cough drop is estimated to be in the range of hundreds of millions of dollars. In addition to sore throat, there is a role for better medication and medication application in the treatment of professional voice users such as actors, singers, lawyers, teachers, etc., as well as in the treatment of patients with postoperative pain, laryngopharyngeal reflux, dysphagia, laryngeal granuloma or radiation induced mucositis. In head and neck cancer alone, the cost of radiation therapy increases by 60% from $14,646 to $20,624 per patient in the presence of mucositis which occurs acutely in 91% of patients. This does not account for chronic xerostomia which has a high morbidity and occurs in 64% of these patients.

A therapy directed at treating the inflammation and dryness of the larynx, vocal folds and cervical trachea as well as optimizing the function of the vocal fold is needed. The currently available lozenges, sprays and gargles are suboptimal as they do not contact or treat the surfaces most contributory to patients' complaints. The inhalant industry focuses on development of inhalers that dispense particles less than 5 microns so the chosen therapeutic can be targeted to the lungs and bypass the upper aero digestive tract. Accordingly, there is a long-felt need in the art for improved inhalant devices.

### SUMMARY

In meeting the described long-felt needs, the present disclosure provides a nebulizer device comprising a housing comprising a mouthpiece and a cover movable between a first position and a second position; a reservoir inside the housing configured to hold a liquid therapeutic formulation; a vibratory mesh nebulizer inside the housing in fluid communication with the reservoir and the mouthpiece, wherein the nebulizer is configured to nebulize the liquid therapeutic formulation into aerosolized particles in a range of 10 to 35 microns; wherein when the cover is in the first position the cover covers the mouthpiece and inactivates the nebulizer and when the cover is in the second position the mouthpiece is exposed to a user and the device is activated to nebulize the liquid therapeutic formulation.

Embodiments include the following, alone or in any combination.

The housing can be generally cylindrical with a radius greater than its height, the mouthpiece is disposed in a recess on a segment of the cylinder's circumference, and the cover is pivotable about an axis to move between the first position and the second position.

The nebulizer device further comprises a battery for powering the device and a printed circuit board inside the housing, wherein the printed circuit board comprises a controller configured to turn on the nebulizer to dispense a dose of the aerosolized particles through the mouthpiece to be inhaled by a user and then turn off the nebulizer when the cover is in the second position.

The nebulizer device is configured to deliver the aerosolized particles to selected areas of the upper airway of the user.

The nebulizer device is configured to deliver the aerosolized particles to the oral cavity, larynx, including the vocal folds, glottis and subglottis, oropharynx, hypopharynx and cervical trachea of the user.

The nebulizer device is configured to deliver the aerosolized particles to surfaces of the larynx, including the vocal folds, glottis and subglottis, and cervical trachea of the user.

The nebulizer device can be configured to nebulize the liquid therapeutic formulation into aerosolized particles in a range of 15 to 30 microns.

The nebulizer device can be configured to nebulize the liquid therapeutic formulation into aerosolized particles in a range of 15 to 25 microns.

The liquid therapeutic formulation can be useful in connection with any one or more of sore throat, throaty cough, xerostomia, pharyngitis, laryngitis, postoperative pain, dysphagia, laryngeal granuloma, vocal optimization, laryngopharyngeal reflux, dysbiosis, laryngeal sensory dysfunction, wound healing, airway stenosis, infection-including any of bacterial, fungal, and viral infection, viral infection prevention, delivery of substances that inhibit viral or bacterial adherence/invasion/infection, delivery of substances that upregulate laryngeal sensation for treatment of issues such as dysphagia and down regulate sensation in laryngeal hypersensitivity disorders. The formulation is useful, for example, in treating sore throat, throaty cough, laryngitis, vocal optimization, laryngopharyngeal reflux, dysbiosis, and laryngopharyngeal sensory dysfunction. The foregoing list of uses is not exhaustive. It should be understood that the disclosed devices can be used to treat a subject's nasal cavity. Without being bound to any particular theory, a subject using a device according to the present disclosure can breathe in through their mouth and then exhale out their nose. In this way, the subject's nasal cavity can be treated.

The formulation can comprise an aqueous solution comprising any one or more active ingredients selected from the group consisting of marshmallow root, slippery elm extract, aloe vera extract, manuka honey, honey, holy basil, menthol, spearmint, capsaicin, propolis, curcumin, xylitol, turmeric, probiotics, and calendula; the formulation can also include any one or more of lidocaine, an antibiotic, an antiviral, and an antifungal medicine. The disclosed formulation can include any one or more of zinc, azadirachtin B, tinosporin, flavonoids, citrus, galangal, Oleanonic acid, angeloylgomisin O, schisandrin B, and procyanidin, curcuma and hydroxytyrosol. The formulation can, in particular, comprise marshmallow root, slippery elm extract, aloe vera extract, and manuka honey. The liquid therapeutic formulation can comprise glycerin. The liquid therapeutic formulation can be buffered to a pH of about 8.3 to about 8.7.

Another aspect provides a formulation useful for treating sore throat, throaty cough, xerostomia, pharyngitis, laryngitis, postoperative pain, dysphagia, laryngeal granuloma, vocal optimization, laryngopharyngeal reflux or dysbiosis wherein the formulation comprises an aqueous solution comprising one or more active ingredients selected from the group consisting of marshmallow root, slippery elm extract, aloe vera extract, manuka honey, honey, holy basil, menthol, spearmint, capsaicin, propolis, curcumin, xylitol, turmeric, probiotics, and calendula nebulized into aerosolized particles in a range of 10 to 35 microns. Notably, the formulation is useful for treating sore throat, throaty cough, laryngitis, vocal optimization, laryngopharyngeal reflux or dysbiosis. The formulation is particularly useful for treating vocal optimization, laryngopharyngeal reflux or dysbiosis. Other uses include treatment of mucositis.

Embodiments of the formulation include the following, alone or in any combination.

The formulation can comprise marshmallow root, slippery elm extract, aloe vera extract, and manuka honey. The formulation can comprise glycerin. The formulation can be buffered to a pH of about 8.3 to about 8.7.

The formulation can comprise aerosolized particles in a range of 20 to 30 microns.

Another aspect provides a method for treating sore throat, throaty cough, xerostomia, pharyngitis, laryngitis, postoperative pain, dysphagia, laryngeal granuloma, vocal optimization, laryngopharyngeal reflux or dysbiosis in a subject, the method comprising providing a solution-which solution can be aqueous or oil-based-comprising one or more active ingredients selected from the group consisting of marshmallow root, slippery elm extract, aloe vera extract, manuka honey, honey, holy basil, menthol, spearmint, capsaicin, propolis, curcumin, xylitol, turmeric, probiotics, and calendula; nebulizing the aqueous solution into aerosolized particles in a range of 10 to 35 microns; and delivering the aerosolized particles to selected areas of the upper airway of the subject. Notably, the formulation is useful for treating sore throat, throaty cough, laryngitis, vocal optimization, laryngopharyngeal reflux or dysbiosis. The formulation is especially useful for treating vocal optimization, laryngopharyngeal reflux or dysbiosis. Other indications include mucositis.

In an embodiment of the method, providing the aqueous solution, nebulizing the aqueous solution and delivering the aerosolized particles are carried out using a nebulizer device comprising a housing comprising a mouthpiece and a cover movable between a first position and a second position; a reservoir inside the housing configured to hold a liquid therapeutic formulation; a vibratory mesh nebulizer inside the housing in fluid communication with the reservoir and the mouthpiece, wherein the nebulizer is configured to nebulize the liquid therapeutic formulation into aerosolized particles in a range of 10 to 35 microns; wherein when the cover is in the first position the cover covers the mouthpiece and inactivates the nebulizer and when the cover is in the second position the mouthpiece is exposed to the subject and the device is activated to nebulize the liquid therapeutic formulation when the mouthpiece of the device is inserted into the oral cavity of the subject. Without being bound to any particular theory or embodiment, a device according to the present disclosure can be configured to maintain the formulation being dispensed nearby to or even in contact with the mesh. This can be accomplished by, for example, use of a dual chamber system, a positive pressure system, or negative pressure system. As an example, one can use a double lumen that includes a comparatively large reservoir and a smaller portion that allows for one-way flow of the formulation against the mesh arranged such that gravity pulls the formula into the smaller portion and encourages the formulation up against the mesh. One can also, for example, use a negative pressure bag located within the reservoir. One can also use a positive pressure-which can be exerted by a mechanical element, such as a spring or elastomer—to encourage the formulation towards or even against the mesh. The reservoir can, for example, be pressurized to encourage movement of the formulation from the reservoir. A device can also be configured so as to effect a negative pressure that encourages movement of the formulation from the reservoir.

In a further aspect, the present disclosure provides a nebulizer device, comprising: a housing comprising a mouthpiece and a cover movable between a first position and a second position, the nebulizer device being configured to contain therein a liquid therapeutic formulation; and a vibratory nebulizer being disposed inside the housing and being in fluid communication with the liquid therapeutic formulation and the mouthpiece, the vibratory nebulizer optionally comprising a mesh, the vibratory nebulizer configured to nebulize the liquid therapeutic formulation into aerosolized particles in a range of 3 to 35 microns, optionally from about 10 to about 35 microns, and the nebulizer device configured such that when the cover is in the first position the cover obstructs the mouthpiece and inactivates the nebulizer and when the cover is in the second position the mouthpiece is exposed to a user.

Also provided is a method, comprising operating a nebulizer device according to the present disclosure-such as according to any one of Aspects 22-40-so as to deliver aerosolized particles of a liquid therapeutic formulation to a subject.

Further provided is a nebulizer device, comprising: a housing configured to contain therein a liquid therapeutic formulation; a vibratory nebulizer disposed within the housing; and the nebulizer device being convertible between (1) a first state in which fluid communication between a subject and the liquid therapeutic formulation is interrupted and (2) a second state in which fluid communication is effected between the subject and the liquid therapeutic formulation and the nebulizer device is activated to nebulize the liquid therapeutic formulation.

Additionally provided is a method, comprising determining an effective aerosol particle size and an effective aerosol velocity. Without being bound to any particular theory or embodiment, the effective aerosol particle size and the velocity of the aerosol can influence drug particle deposition in the larynx.

### BRIEF DESCRIPTION OF THE DRAWINGS

In order that the advantages of the device will be readily understood, a more particular description of the device briefly described above will be rendered by reference to specific embodiments that are illustrated in the appended drawings. Understanding that these drawings depict only typical embodiments of the device and are not therefore to be considered as limiting of its scope, the device and methods for using it will be described and explained with additional specificity and detail using the accompanying drawings.

FIGS. 1-8 provide example aspects of a simulated nebulized drug delivery in the airway, according to an exemplary embodiment of the disclosed subject matter.

FIGS. 9-16 depict an exemplary, non-limiting device for administering nebulized particles to the airway of a subject, according to an exemplary embodiment of the disclosed subject matter.

### DETAILED DESCRIPTION OF ILLUSTRATIVE EMBODIMENTS

The present disclosure may be understood more readily by reference to the following detailed description of desired embodiments and the examples included therein.

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing. All publications, patent applications, patents and other references mentioned herein are incorporated by reference in their entirety. The materials, methods, and examples disclosed herein are illustrative only and not intended to be limiting.

The singular forms “a,” “an,” and “the” include plural referents unless the context clearly dictates otherwise.

As used in the specification and in the claims, the term “comprising” can include the embodiments “consisting of” and “consisting essentially of.” The terms “comprise(s),” “include(s),” “having,” “has,” “can,” “contain(s),” and variants thereof, as used herein, are intended to be open-ended transitional phrases, terms, or words that require the presence of the named ingredients/steps and permit the presence of other ingredients/steps. However, such description should be construed as also describing compositions or processes as “consisting of” and “consisting essentially of” the enumerated ingredients/steps, which allows the presence of only the named ingredients/steps, along with any impurities that might result therefrom, and excludes other ingredients/steps.

As used herein, the terms “about” and “at or about” mean that the amount or value in question can be the value designated some other value approximately or about the same. It is generally understood, as used herein, that it is the nominal value indicated ±10% variation unless otherwise indicated or inferred. The term is intended to convey that similar values promote equivalent results or effects recited in the claims. That is, it is understood that amounts, sizes, formulations, parameters, and other quantities and characteristics are not and need not be exact, but can be approximate and/or larger or smaller, as desired, reflecting tolerances, conversion factors, rounding off, measurement error and the like, and other factors known to those of skill in the art. In general, an amount, size, formulation, parameter or other quantity or characteristic is “about” or “approximate” whether or not expressly stated to be such. It is understood that where “about” is used before a quantitative value, the parameter also includes the specific quantitative value itself, unless specifically stated otherwise.

Unless indicated to the contrary, the numerical values should be understood to include numerical values which are the same when reduced to the same number of significant figures and numerical values which differ from the stated value by less than the experimental error of conventional measurement technique of the type described in the present application to determine the value.

All ranges disclosed herein are inclusive of the recited endpoint and independently of the endpoints. The endpoints of the ranges and any values disclosed herein are not limited to the precise range or value; they are sufficiently imprecise to include values approximating these ranges and/or values.

As used herein, approximating language can be applied to modify any quantitative representation that can vary without resulting in a change in the basic function to which it is related. Accordingly, a value modified by a term or terms, such as “about” and “substantially,” may not be limited to the precise value specified, in some cases. In at least some instances, the approximating language can correspond to the precision of an instrument for measuring the value. The modifier “about” should also be considered as disclosing the range defined by the absolute values of the two endpoints. For example, the expression “from about 2 to about 4” also discloses the range “from 2 to 4.” The term “about” can refer to plus or minus 10% of the indicated number. For example, “about 10%” can indicate a range of 9% to 11%, and “about 1” can mean from 0.9-1.1. Other meanings of “about” can be apparent from the context, such as rounding off, so, for example “about 1” can also mean from 0.5 to 1.4.

Further, the term “comprising” should be understood as having its open-ended meaning of “including,” but the term also includes the closed meaning of the term “consisting.” For example, a composition that comprises components A and B can be a composition that includes A, B, and other components, but can also be a composition made of A and B only. Any documents cited herein are incorporated by reference in their entireties for any and all purposes.

Any embodiment or aspect provided herein is illustrative only and does not limit the scope of the present disclosure or the appended claims. Any part or parts of any one or more embodiments or aspects can be combined with any part or parts of any one or more other embodiments or aspects.

In accordance with various embodiments of the invention, and as shown in the figures, various devices and methods are disclosed that provide selective deposition of aerosolized particles to areas of the upper airway of a subject.

Disclosed herein is a low-cost portable disposable inhalant delivery device that targets a liquid therapeutic formulation to the larynx, including the vocal folds, glottis and subglottis, and cervical trachea as well as the oral cavity, oropharynx and hypopharynx for the treatment of sore throats, throaty cough, laryngopharyngeal reflux and xerostomia as well as the optimization of vocal fold function for healthy professional voice users such a singers, actors, teacher, lawyers and customers service personnel. Inflammation of the larynx, vocal folds and trachea leads to throat pain and irritation, hoarseness, and coughing. These areas are not treated with the typical over the counter medications, gargles, sprays and lozenges, as they do not contact the areas mentioned at all and only treat localized areas of the oral cavity and pharynx. While there are many inhalation devices that deliver medication to the lungs, there is a lack of inhalation devices that target delivery of therapeutic agents to the larynx, including the vocal folds, glottis and subglottis, and upper trachea to treat sore, dry, and irritated throats and provide phonatory optimization. This can warrant use of a specific delivery device to produce nebulized particles of a specific size. Without being bound to any particular theory or embodiment, such a delivery device can be a portable, self-contained system that allows for easier access to treatment based on the ease of storing such a device as a one-piece system. Existing portable nebulizers frequently include add-on mouth pieces, which can complicate storage, use, and portability.

As used herein, a liquid therapeutic formulation is a formulation comprising at least one active ingredient that provides biologically active or other direct effect in the diagnosis, cure, mitigation, treatment, or prevention of disease or to affect the structure or any function of the body of a living subject, optionally including an additional ingredient selected from solvents, excipients, diluents, surfactants, pH modifiers, solubilizers, stabilizers, buffers, tonicity modifiers, bulking agents, viscosity enhancers/reducers, surfactants, chelating agents, adjuvants, flavorants or taste maskers. Active ingredients can include, as but some examples, any one or more of natural products, plant extracts, biologics, probiotics, prescription or nonprescription analgesics, anesthetics, antibiotics, antivirals, antifungals, and the like.

The inhalation device described herein would evenly deliver therapeutics to the larynx, including the vocal folds, glottis and subglottis, and upper trachea as well as the oral cavity, oropharynx, and hypopharynx. The main body of the device has an enclosed chamber or reservoir for the therapeutic formula in contact with a mesh specifically developed to nebulize the formula into 10 to 35 micron particles. Together they work as one unit specifically developed to release the specific micron range to target the larynx and trachea. There is an open end to emit the mist from the chamber. The mist travels through a specifically shaped and designed mouthpiece to minimize loss of therapeutic agent to allow specific targeting of the vocal folds and cervical trachea as well as the oral cavity and pharynx. There is a movable cover over the mouthpiece that keeps the mouthpiece clean. Moving or opening the cover allows access of the user to the mouthpiece and turns on the device, which activates the mesh and dispenses the mist for inhalation for 10-30 seconds until deactivation. While on, an optional LED lights to show that the device is nebulizing particles.

The mesh nebulizer described herein requires far less time to atomize medication than conventional systems. The high-frequency operation is noiseless for humans and the low energy requirement of the piezo components enables their use in a battery-powered inhalation device described herein. This means that the device is portable and can be conveniently used when needed.

The disclosed inhalation devices can be configured to deliver naturopathic or allopathic medication to the structures that need it and be more effective for the treatment of a variety of condition. Some examples conditions include sore throat, throaty cough, laryngitis, vocal optimization, laryngopharyngeal reflux or dysbiosis, as well as other indications. It should be understood that the foregoing list is illustrative and is not limiting or exhaustive.

In an embodiment, the device would deliver a therapeutic agent in mist comprising a combination of active ingredients selected from the group consisting of marshmallow root, slippery elm extract, aloe vera extract, manuka honey, honey, holy basil, menthol, spearmint, capsaicin, propolis, curcumin, xylitol, turmeric, probiotics, and calendula. The formulation is buffered to a specified pH range. Other formulation ingredients include glycerin and sucralose. Optionally, additional agents in the formulation include flavorings, such as natural cherry, mint or other flavors.

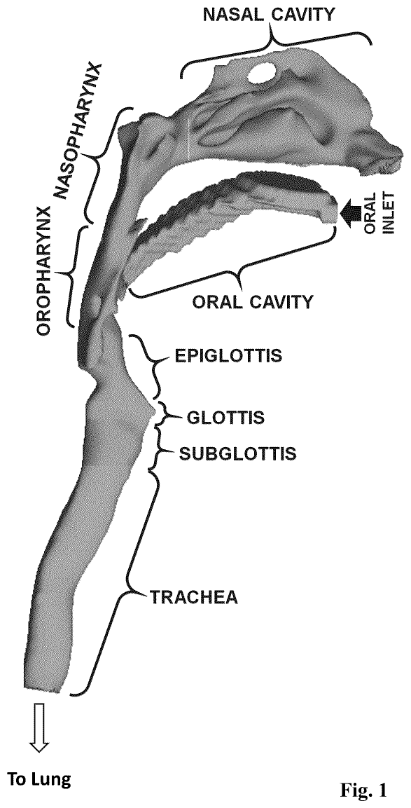

FIG. 1 shows a cross-section of a human airway. Air can be inhaled through the oral cavity (mouth) or nasal cavity (nose) and enters the pharynx, comprising the nasopharynx, oropharynx and hypopharynx. In the middle of the hypopharynx is the larynx (voice box). The larynx includes the supraglottis, glottis (passage between the vocal folds), and subglottis. Within these three regions the cartilage, neurovascular, and musculature are all intertwined and interactive to allow the larynx to function as a unit and carry out its many functions. The primary functions of the larynx are voice production, protection of the airway during respiration, and swallowing. The vocal folds, also called vocal cords, are two bands of muscle and soft tissue found in the larynx. The vocal folds lie in the center of the larynx in a front to back alignment. The epiglottis lies on the top portion of the larynx and protects the larynx during swallowing and prevents aspiration (breathing in) of food. The epiglottis is a flap of soft tissue and cartilage located just above the vocal cords. The epiglottis folds down over the vocal cords to help prevent food and irritants from entering the lungs. The trachea lies below the larynx and extends the airway into the lungs (not shown).

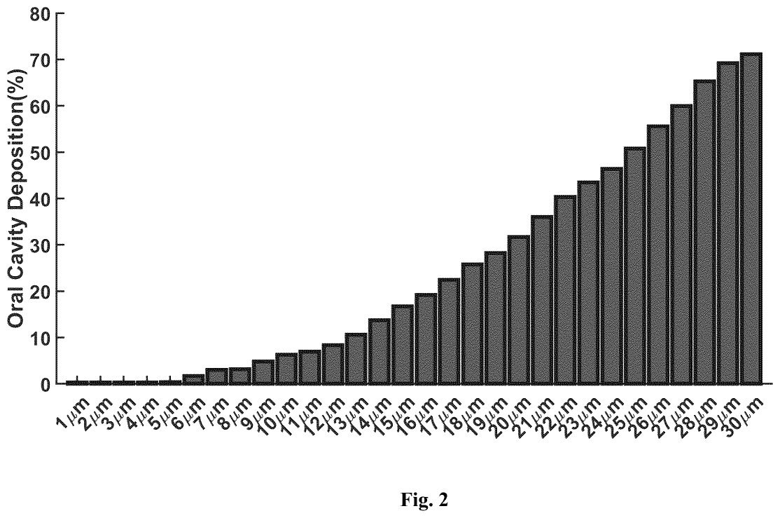

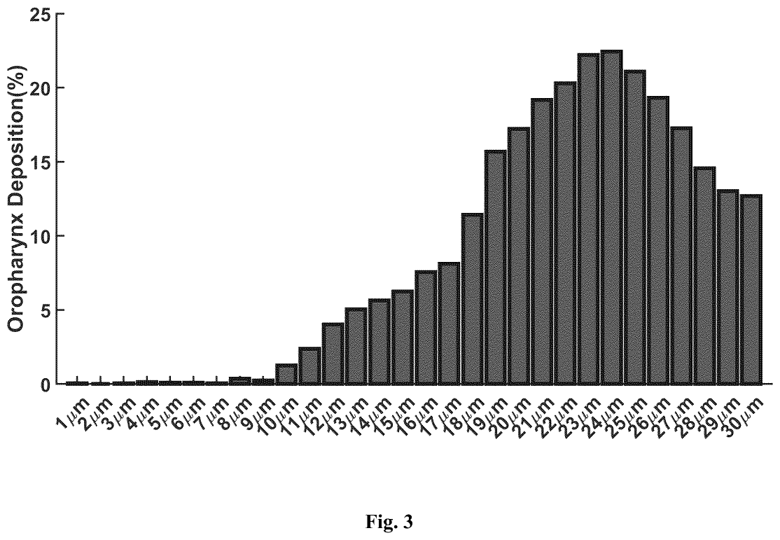

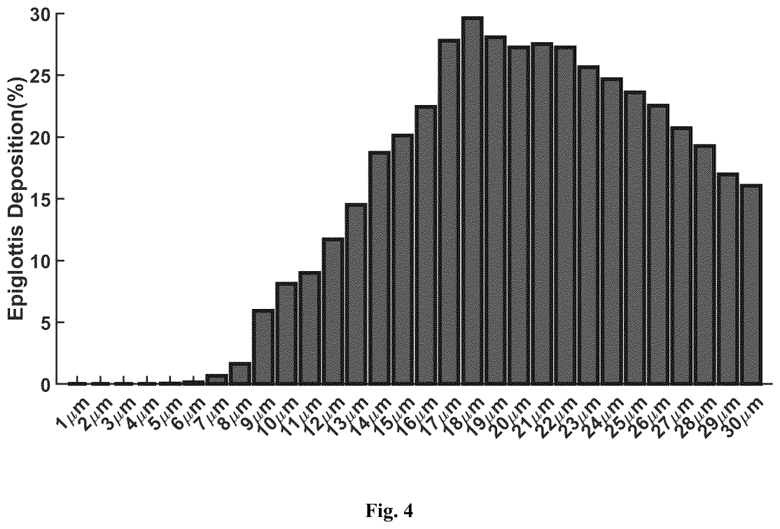

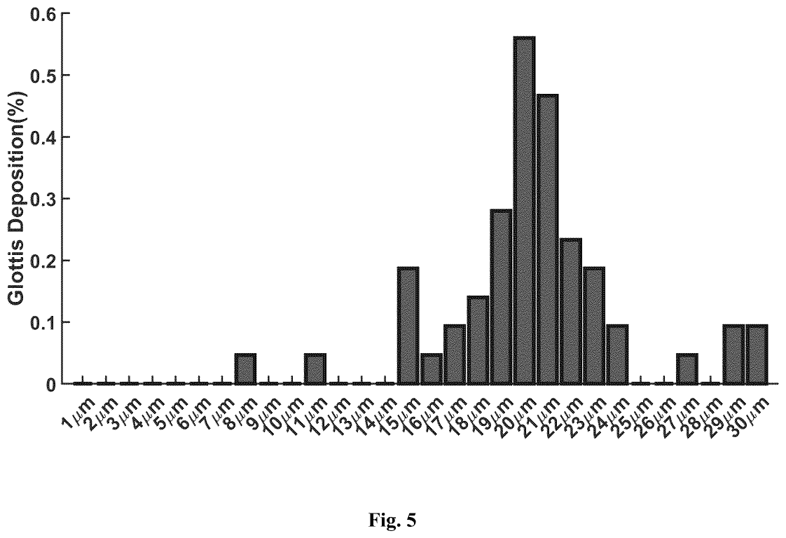

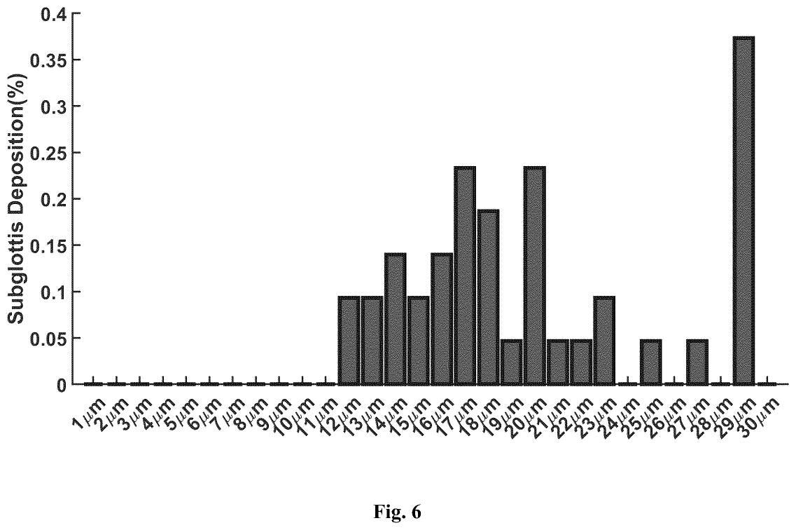

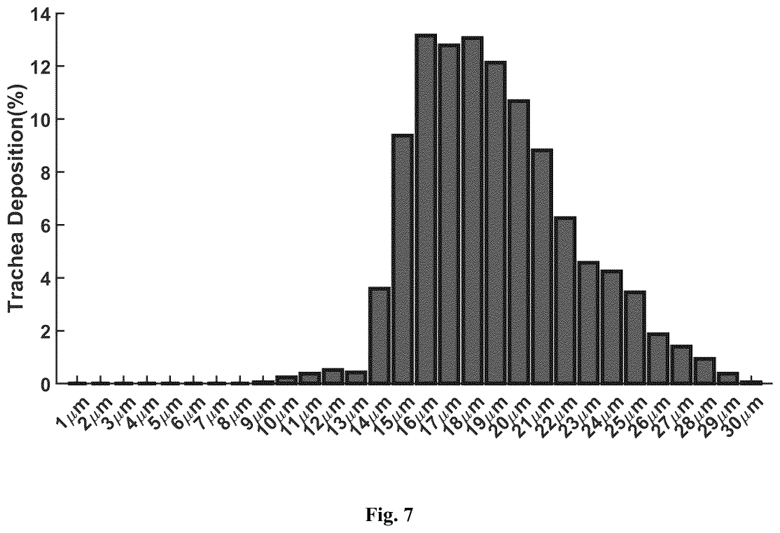

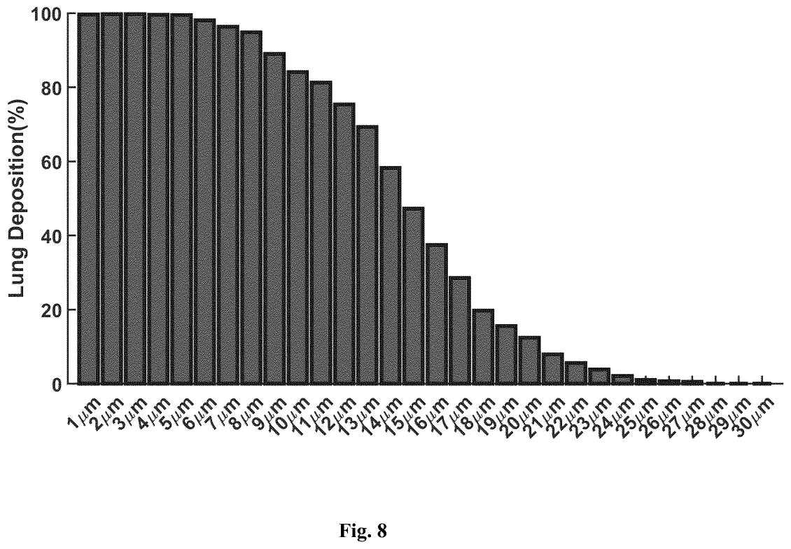

FIGS. 2 through 8 show the results of a computer simulated inhalation of nebulized particles through the airway and where the particles are deposited. The simulated flow rate through the airway was based on a resting inspiration at 15 L/min. The simulated nebulized particles were released from the oral inlet with particle velocity of 1 m/s. The particle diameters ranged from 1 μm for 30 μm, with 1 μm increments. Percentage of depositions were calculated for the following regions (see FIG. 1): Oral cavity, Oropharynx, Epiglottis, Glottis, Subglottis, Trachea, and Lung (not shown in FIG. 1). The results are shown in FIGS. 2-8.

FIGS. 2-8 show that the regions of deposition are highly dependent on particle size. Particles below about 10 μm are deposited primarily in the lung, while particles above 30 μm are deposited primarily in the oral cavity. Particles in the range of about 10 μm to about 30 μm or about 10 μm to about 30 μm, such as about 15 μm to about 30 μm, about 20 μm to about 30 μm, about 15 μm to about 25 μm, about 15 μm to about 20 μm, or about 20 μm to about 25 μm can be selectively deposited in regions of the throat. One can appreciate that medicaments in particles deposited on the oropharynx and epiglottis could flow downward toward the glottis and vocal folds.

A device according to an embodiment of the disclosed subject matter is depicted in the following Figures.

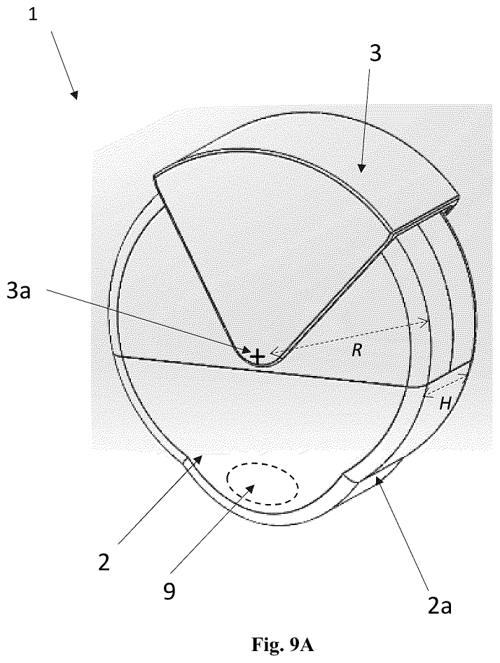

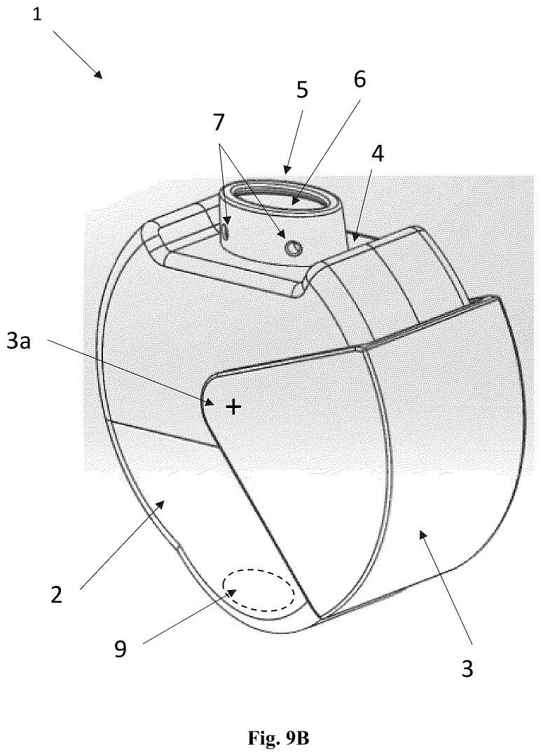

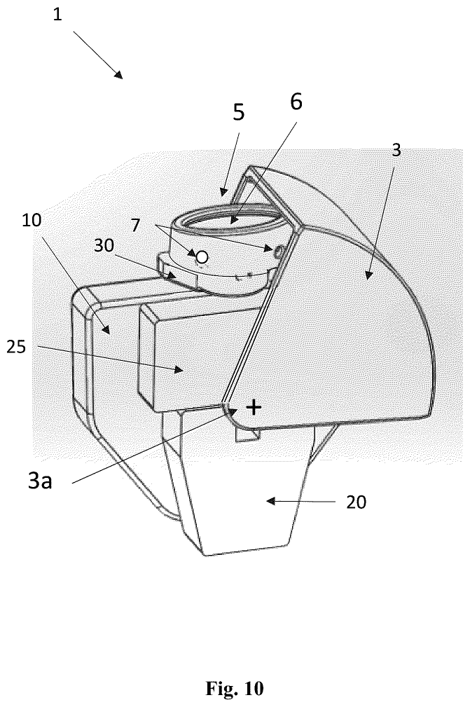

FIGS. 9A and 9B show an exemplary device 1 for delivering nebulized particles to a subject's upper airway. The device shown is generally disk-shaped, comprising a generally cylindrical housing 2. The housing 2 has a radius R greater than its height H. It can be sized to fit in a pocket or purse. For example, an embodiment can have a nominal diameter of 60-90 mm, such as 76 mm, and a height (thickness) of 20 to 40 mm, such as 36 mm. A wedge-shaped cover 3 is pivotable or rotatable about axis 3a to move from a first position (as shown in FIG. 9A) wherein the cover closes the device and covers a mouthpiece of the device. FIG. 9B shows the cover 3 rotated to the second position, which exposes the mouthpiece 5 and activates the device. An edge 2a on the housing 2 provides a stop to prevent over-rotation of the cover and allows the fluid reservoir 10 (see FIG. 10) to be somewhat larger. A recess 4 in the housing 2 provides space for the mouthpiece 5 to be covered when the cover 3 is in the first, closed position. The mouthpiece 5 comprises an outlet 6 for a subject to orally inhale the medicament when the device is activated. Outlet hole(s) 7 on the perimeter of the mouthpiece allow the subject to exhale while holding the device in their mouth. The mouthpiece can be removable for cleaning or replacement.

The device is activated when the cover 3 is moved to the second position. Activation means that the cover completes a circuit by, for example, a magnetic switch and turns on a piezoelectric nebulizer mesh, described further below.

Optionally, the device comprises a light-emitting diode (LED) 9 that lights when the device is activated by moving the cover to the second, activating position. For example, the LED can flash blue, green or purple (or other color) when the device is nebulizing the medicament. It can change color or emit steadily when the dose is done. The LED is disposed on the top of the device in an area not covered by the open cover, so a user can see it as they inhale. In embodiments, the LED can indicate when the medicament chamber is empty when the cover is moved to the activating position. For example, the LED can indicate red when there is no medicament left.

FIG. 10 shows the device with the housing 2 removed to show the internal components of the device. A chamber or reservoir 10 holds a solution of the medicament. In an example embodiment, reservoir 10 can hold up to 30 ml of therapeutic solution. A printed circuit board (PCB) 20 includes the electrical and electronic components needed to operate the device. For example, the PCB can comprise one or more controllers, memory and other components. A battery 25 powers the device.

In some embodiments for a “one-time” use, the reservoir 10 can be sealed within the housing and contains enough doses of the medicament for a user to treat a condition and then discard the device. This, however, is not a requirement, as the housing can itself contain the medicament without the presence of a separate reservoir. It can be appreciated that the battery 25 can be appropriately sized to have a charge sufficient to deliver the doses in the device. Such embodiments can be useful to a user to buy the device over the counter to treat a transient illness such as a cold or flu. In other instances, these embodiments can be used to deliver a limited number of doses of a prescribed medicament (for example lidocaine) to treat an acute condition and/or a chronic condition. Example conditions include, without limitation, xerostomia, radiation induced mucositis, or dysphagia. The disclosed device can be used, as an example, for delivery of probiotics for treatment of dysbiosis.

In other embodiments for a reusable device, the housing can be configured so as to allow the reservoir 10 to be replaced or refilled with additional or alternate formulations. In these embodiments, the battery 25 can also be rechargeable or replaceable and/or the mouthpiece can be removed for cleaning or replacement. These embodiments can be envisioned for long-term use.

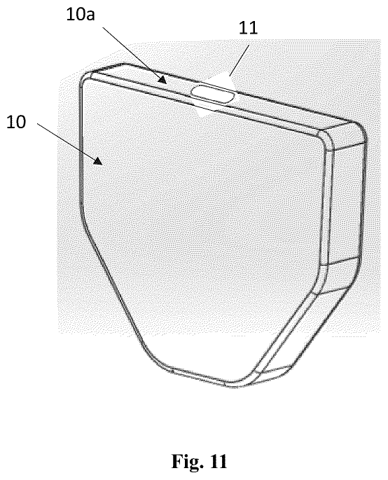

FIG. 11 shows a view of the reservoir 10. An opening 11 on the edge 10a provides a passage from the reservoir to the nebulizer portion of the device for the therapeutic solution.

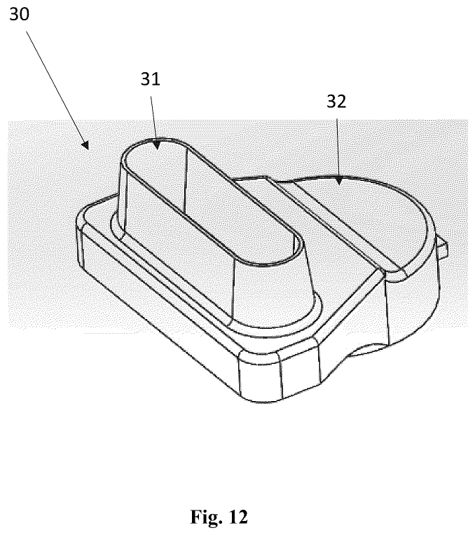

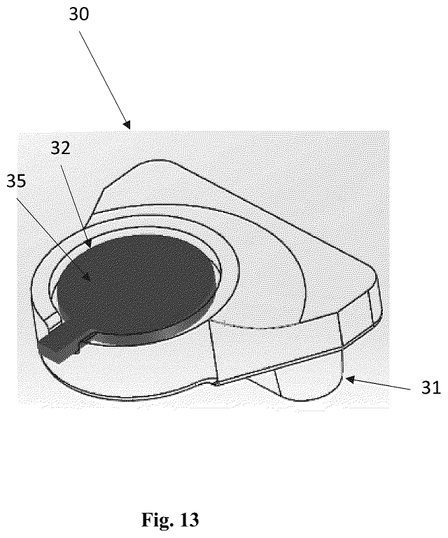

FIG. 12 shows a funnel or passage 30 that allows medicament to move from the reservoir 10 to a piezoelectric vibratory mesh to be nebulized. First open end 31 is inserted into opening 11 on the reservoir and the passage turns about 90° and spreads out into a generally cylindrical second end 32, which is engaged to the piezoelectric vibratory mesh (see FIG. 13). As described elsewhere herein, a device according to the present disclosure can be configured to maintain the formulation being dispensed nearby to or even in contact with the mesh. This can be accomplished by, for example, use of a dual chamber system, a positive pressure system, or negative pressure system. As an example, one can use a double lumen that includes a comparatively large reservoir and a smaller portion that allows for one-way flow of the formulation against the mesh arranged such that gravity pulls the formulation into the smaller portion and encourages the formulation up against the mesh. One can also, for example, use a negative pressure bag located within the reservoir. One can also use a positive pressure-which can be exerted by a mechanical element, such as a spring or elastomer—to encourage the formulation towards or even against the mesh. The reservoir can, for example, be pressurized to encourage movement of the formulation from the reservoir. A device can also be configured so as to effect a negative pressure that encourages movement of the formulation from the reservoir.

FIG. 13 shows the funnel 30 from the opposite side. Second end 32 has a round recess that engages the disk-shaped piezoelectric vibratory mesh 35 overlaying an opening (not visible) in the second end 32.

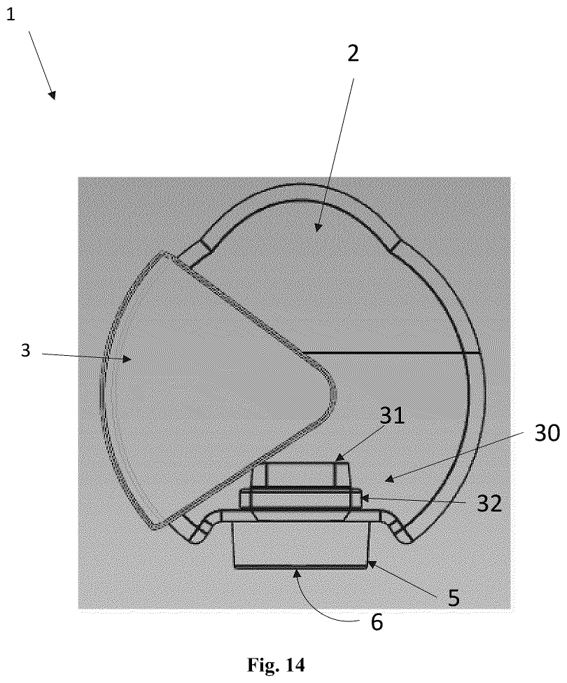

FIG. 14 shows the funnel 30 inside the housing 2 (transparent in this view) to show how it is positioned next to the mouthpiece 5. Second end 32 holds the piezoelectric mesh disk 35 against the housing 2 in fluid communication with the air passage in the mouthpiece.

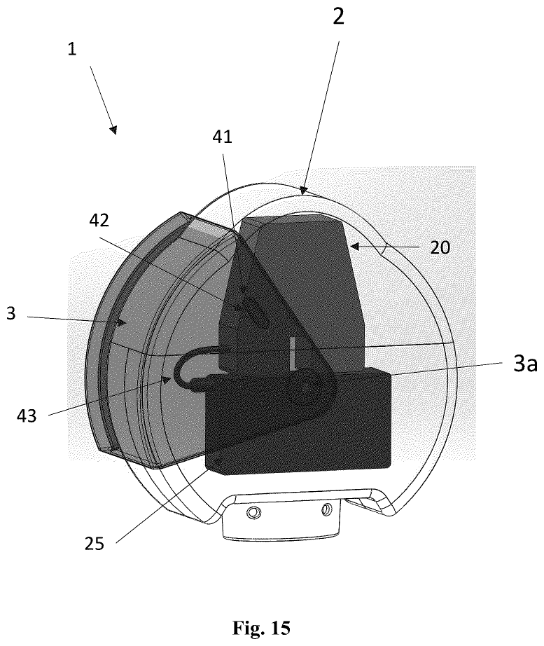

FIG. 15 shows the device with the cover and housing depicted as transparent to allow visualization of the electrical components inside the device. Printed circuit board 20 is connected to the battery 25 with a power connector 43. A magnetic surface on the inside face of the cover 3 is configured to interact with a magnetic surface 42 on the PCB when the cover 3 is moved to the second or open position. When 41 and 42 are aligned, the device is activated to operate the mesh nebulizer and nebulize the medicament for delivery to the user.

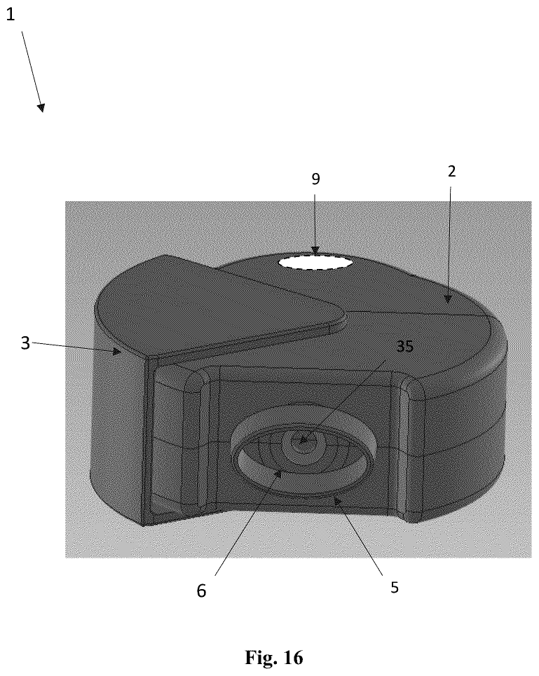

FIG. 16 shows an exterior view of the device 1 when ready to be used to dispense a dose of the therapeutic liquid. The device 1 is disposed with the mouthpiece 5 toward the user in a generally horizontal orientation. The cover 3 is in the second, open position to expose the mouthpiece 5. The piezoelectric mesh disk 35 is visible through the opening 6 in mouthpiece 5. The optional LED 9 is shown, which would be visible to a user when inhaling through the device. The LED 9, or some other indicator, shows the top of the device in use, such that the piezoelectric disk is located below the mouthpiece.

Piezo atomizers or nebulizers are used to atomize liquid, creating and dispensing mist. The fluid solution is placed in reservoir 10 (not shown in this figure, see FIG. 10) in fluid communication with a piezo element, piezo disk 35 via funnel 30. Piezo disk 35 is a perforated mesh disk with several thousands of holes. When an electrical voltage is applied to the piezo atomizer, the piezo element generates ultrasonic frequencies. In the mesh nebulizer 35, specially shaped piezo rings act as ultrasonic transducers and excite the metal membrane to high-frequency vibrations. The rapid ultrasonic vibration from the piezoceramic causes the fluid to be pushed through the mesh disk, producing tiny droplets or mist. The vibrations produce particularly homogenous aerosols without heating up the medication. In the process, the therapeutic components are only subjected to low shear forces since they pass the membrane only once during atomization. The micron-size droplets produced by the piezoelectric atomizer are extremely uniform in size and distribution due to the mesh disk. The number of holes and size of holes in the mesh disk is a key factor in determining the droplet size and flow rate. The size of the holes and vibratory frequency are configured to produce aerosols with particle sizes in the range of about 10 μm to about 35 μm, such as about 20 μm to about 30 μm to be selectively deposited in regions of the throat.

As discussed, moving the cover 3 to the second open position exposes the mouthpiece 5 and activates the device by tripping a switch. Activating the device causes a controller in the PCB to turn on the nebulizer to dispense a dose of therapeutic mist through the mouthpiece to be inhaled by a user and then turn off the nebulizer. The controller also controls the emission format of the LED 9, if present, to provide a visual indicator of the status of the device. In a specific embodiment, the nebulizer device 1 dispenses micron-sized particles at a rate of 0.02 ml/sec. for 20 seconds, or about 0.4 ml of fluid per dose. The particles can be, for example but not limitation. 15 to 25 microns, or 20-30 microns. However, delivery of that dosage is not limiting and can be modified depending on what therapeutic is to be delivered by the device. In embodiments, the rate of aerosol production can be the same, but the duration of delivery can be adjusted higher or lower by programming the PCB controller. In embodiments, the mesh size can be switched to a different size and/or the vibration frequency can be changed to produce particles with a different size distribution.

In the specific embodiment shown in the figures, the reservoir 10 has a capacity of about 30 ml, or about 75 doses of 0.4 ml each. Using the device 3 to 4 times a day for two weeks would represent a maximum of 56 doses.

To use the device, the user moves the cover from the closed position to the open position and inserts the mouthpiece into his mouth. Aerosolized mist is dispensed to the user's mouth as he respires orally for the predetermined dosage time. When the time expires, the controller stops the nebulization and optionally changes the LED display to indicate the dose is finished. The user removes the device from his mouth and closes the cover until another dose is indicated.

In embodiments, the device can deliver a therapeutic agent in mist comprising a combination of active ingredients selected from the group consisting of marshmallow root, slippery elm extract, aloe vera extract, manuka honey, honey, holy basil, menthol, spearmint, capsaicin, propolis, curcumin, xylitol, turmeric, probiotics, and calendula. A notable combination includes marshmallow root, slippery elm extract, aloe vera extract, and manuka honey. For example, the formulation can be an aqueous solution including aloe, manuka honey, marshmallow, and slippery elm in a ratio of about 10:1:0.26:0.26. The solution can comprise around 1% of xylitol for sweetening while preventing dental caries. Xylitol also increases saliva production for improving throat moisture. The formulation can be buffered to a specified pH range around 8.5 to denature pepsin for treatment of laryngopharyngeal reflux. Other formulation ingredients include glycerin and sucralose. Optionally, additional agents in the formulation include flavorings, such as natural cherry, mint or other flavors.

An example formulation is designed to alleviate or treat sore throat symptoms, optimize phonation, and treat laryngopharyngeal reflux. The viscosity was chosen so the formula would work with a mesh nebulizer to produce the desired particle size and is similar to the viscosity of saline. The formula was created to have a specific osmolarity that would be hydrating and hypotonic, with osmolarity in a range of 200 to 250 mOsm/kg, compared to that of saline (308 mOsm/kg).

Example 1 was chosen for a formulation because its appearance, pH, and osmolarity were physically stable for 31 days at 25° C.

The chemical stability of Example 1 was not evaluated. Allopathic medications could also be delivered via this device for more severe mucosal disorders and/or increased potency, post-operative surgical pain or laryngeal granulomas. Treatment of dysphagia/swallowing problems can be another application of the device with a different formula.

### EXAMPLES

### Materials

### Example 1

TABLE 1
Materials for manufacturing Example 1 Formulation.

| | | | Target |
|---|---|---|---|
| | | | Composition |
| Name | CAS Number | Function | (% w/v) |
| Water for Injection | 7732-18-5 | Solvent | 96.9137 |
| Xylitol | 87-99-0 | Sweetener | 1.0000 |
| Phenoxyethanol | 122-99-6 | Preservative | 0.8000 |
| Glycerin | 56-81-5 | Humectant | 0.5000 |
| Sucralose | 56038-13-2 | Sweetener | 0.4500 |
| Aloe Barbadenis Leaf Extract | 94349-62-9 | Active | 0.1000 |
| | | Ingredient | |
| Disodium EDTA | 139-33-3 | Chelation | 0.1000 |
| | | agent | |
| Tromethamine (Tris Base) | 77-86-1 | Buffer | 0.1211 |
| Honey (Manuka) | 8028-66-8 | Active | 0.0100 |
| | | Ingredient | |
| Athaea Officinalis (Marshmallow) | 73049-65-7 | Active | 0.0026 |
| Root Extract | | Ingredient | |
| Ulmus Fulva (slippery elm) Bark | 90028-27-6 | Active | 0.0026 |
| Extract | | Ingredient | |
| Sodium Hydroxide (5.0N) | 1310-73-2 | pH modifier | As required |
| Hydrochloric Acid (6.0N) | 7647-01-0 | pH modifier | As required |

### Procedure for Preparing a 100 mL Batch of Example 1

Prepare a diluted honey solution by adding 250±50 mg of Manuka honey to a 50 mL volumetric flask. Fill the volumetric flask to the 50.0 mL mark with water for injection (WFI). Calculate the concentration of the diluted honey solution.

Weigh the dry ingredients listed in Table 2 and transfer into beaker. Add 70 mL of WFI to the beaker and stir on a stir plate. Do not proceed to the next step until the solids are fully solubilized.

TABLE 2
Dry ingredients for a 100 mL batch

| Material | Quantity | |
|---|---|---|
| Xylitol | 1000 ± 20 | mg |
| Sucralose | 450 ± 9 | mg |
| Aloe Barbadenis Leaf Extract | 100 ± 2 | mg |
| Disodium EDTA | 100 ± 2 | mg |
| Tromethamine (Tris Base) | 121.14 ± 2.42 | mg |

While stirring, add the liquid ingredients listed in Table 3 to the beaker. Calculate the amount of diluted manuka honey to add to the beaker by using the equation below and the diluted honey concentration calculated in step 1. The amount of diluted honey should be approximately 2000 μL. After adding the liquid ingredients, stir for an additional 15 minutes.

$${V}_{o} = \frac{V * C * {10}^{6}}{{C}_{o}}$$

where:
Vo=Volume of diluted honey solution to add to the beaker in μL.
Co=Concentration of diluted honey solution (5 mg/mL)
V=Volume of Formulation in (100 mL)
C=Target concentration of honey in final formulation (0.01% w/v)

TABLE 3
Liquid ingredients for a 100 mL batch

| | |
|---|---|
| Material | Quantity |

| Phenoxyethanol | 727.27 | μL |
|---|---|---|
| Glycerin | 396.83 | μL |
| Diluted Honey (Manuka)* | Calculated | |
| Athaea Officinalis (Marshmallow) root extract | 2.29 | μL |
| Ulmus Fulva (slippery elm) bark extract | 2.09 | μL |

Measure and adjust the pH of the solution to 8.5±0.25 pH using IN NaOH and IN HCL. Record additions of acid and base.

Transfer the solution into a 100 mL volumetric flask and make to volume with WFI. Cap the flask and invert to mix.

Measure the pH of the solution. If the solution is not to 8.5±0.25 pH, adjust the pH using IN NaOH and IN HCL.

Filter the solution using a 0.22 μm PVDF unit filter and a vacuum source. The collected filtrate is the Example 1 formulation.

Example 1 was characterized by recording the appearance, pH, osmolarity, and viscosity (Table 4). Aliquots of Example 1 were stored in a 25° C. temperature-controlled chamber and characterized at 7 and 31 days after manufacture. Five mL of Example 1 were aliquoted into 2, type 1 glass vials then stored in the chamber. 10 mL of Example 1 were aliquoted into a polypropylene (PP) centrifuge tube then stored in the chamber. The samples in the glass vials were characterized at 7 and 31 days from manufacture. The PP centrifuge tube sample was characterized 31 days from manufacture. The physical characteristics of the PP centrifuge tube and glass vials were not significantly different.

The appearance of Example 1 was evaluated to determine if the formulation precipitated. After manufacturing Example 1, the formulation was inspected for turbidity and visible particles. Example 1 was clear, colorless, and did not show signs of precipitation at the time of manufacture nor 31 days from manufacture.

pH

Example 1 was adjusted to 8.5±0.25 pH using IN sodium hydroxide (NaOH) and IN hydrogen chloride (HCl). The pH values of Example 1 at 0, 7, and 31 days were 8.55, 8.45, and 8.43 pH, respectively (Table 4). Tromethamine (tris base) was used to buffer the formulation and was successful in resisting pH change.

### Osmolality

Osmolality was determined by a freezing point depression osmometer. The osmolality of formulation Example 1 at 0 and 31 days was 223 mOsm/kg. The osmolality of Example 1 did not change after 31 days.

### Viscosity

The viscosity of formulation Example 1 was 0.97 cP. The viscosity of Example 1 was not measured at the 7- and 31-day timepoints.

### Nebulizer Compatibility

Aerosolization of Example 1 was tested using a nebulizer device according to embodiments herein. These tests showed that Example 1 was aerosolizable by the nebulizer.

TABLE 4
Physical characterization of Example 1 at
0, 7, and 31 days after manufacturing.

| Timepoint | | | |
|---|---|---|---|
| (days) | Container | Parameter | Result |
| 0 | N/A | Appearance | Clear, |
| | | | Colorless |
| | | pH | 8.55 |
| | | Osmolarity | 232 |
| | | | mOsm/kg |
| | | Viscosity | 0.97 cP |
| 7 | Sealed, Type 1 Glass | Appearance | Clear, |
| | vial | | Colorless |
| | | pH | 8.45 |
| 31 | Sealed, Type 1 Glass | Appearance | Clear, |
| | vial | | Colorless |
| | | pH | 8.43 |
| | | Osmolarity | 223 |
| | | | mOsm/kg |
| | PP Centrifuge Tube | Appearance | Clear, |
| | | | Colorless |
| | | pH | 8.40 |
| | | Osmolarity | 224 |
| | | | mOsm/kg |

### ASPECTS

The following Aspects are illustrative only and do not limit the scope of the present disclosure or the appended claims. Any part or parts of any one or more Aspects can be combined with any part or parts of any one or more other Aspects.

Aspect 1. A nebulizer device, comprising: a housing comprising a mouthpiece and a cover movable between a first position and a second position, the housing defining a perimeter; a reservoir inside the housing configured to hold a liquid therapeutic formulation; a vibratory mesh nebulizer inside the housing and in fluid communication with the reservoir and the mouthpiece, wherein the nebulizer is configured to nebulize the liquid therapeutic formulation into aerosolized particles in a range of 3 to 35 microns, optionally from about 10 to about 35 microns; wherein when the cover is in the first position the cover covers the mouthpiece and inactivates the nebulizer and when the cover is in the second position the mouthpiece is exposed to a user and the device is activated to nebulize the liquid therapeutic formulation.

Aspect 2. The nebulizer device of Aspect 1, wherein the housing is generally cylindrical with a radius greater than its height, the mouthpiece is disposed in a recess on a segment of the perimeter of the housing, and the cover is pivotable about an axis to move between the first position and the second position.

Aspect 3. The nebulizer device of Aspect 1, further comprising a battery for powering the device and a printed circuit board inside the housing, wherein the printed circuit board comprises a controller configured to turn on the nebulizer to dispense a dose of the aerosolized particles through the mouthpiece to be inhaled by a user and then turn off the nebulizer when the cover is in the second position.

Aspect 4. The nebulizer device of Aspect 1, configured to deliver the aerosolized particles to selected areas of the upper airway of the user.

Aspect 5. The nebulizer device of Aspect 4, configured to deliver the aerosolized particles to the oral cavity, larynx, including the vocal folds, glottis and subglottis, oropharynx, hypopharynx and cervical trachea of the user.

Aspect 6. The nebulizer device of Aspect 4, configured to deposit the aerosolized particles on surfaces of the oral cavity, oropharynx and epiglottis of the user.

Aspect 7. The nebulizer device of Aspect 4, configured to deliver the aerosolized particles to surfaces of the larynx, including the vocal folds, glottis and subglottis, and cervical trachea of the user.

Aspect 8. The nebulizer device of Aspect 1, configured to nebulize the liquid therapeutic formulation into aerosolized particles in a range of 15 to 30 microns.

Aspect 9. The nebulizer device of Aspect 1, configured to nebulize the liquid therapeutic formulation into aerosolized particles in a range of 15 to 25 microns.

Aspect 10. The nebulizer device of Aspect 1, wherein the liquid therapeutic formulation is useful for treating sore throat, throaty cough, xerostomia, pharyngitis, laryngitis, postoperative pain, dysphagia, laryngeal granuloma, vocal optimization, laryngopharyngeal reflux or dysbiosis.

Aspect 11. The nebulizer device of Aspect 1, wherein the liquid therapeutic formulation comprises an aqueous solution comprising one or more active ingredients selected from the group consisting of marshmallow root, slippery elm extract, aloe vera extract, manuka honey, honey, holy basil, menthol, spearmint, capsaicin, propolis, curcumin, xylitol, turmeric, probiotics, and calendula.

Aspect 12. The nebulizer device of Aspect 1, wherein the liquid therapeutic formulation comprises marshmallow root, slippery elm extract, aloe vera extract, and manuka honey.

Aspect 13. The nebulizer device of Aspect 11, wherein the liquid therapeutic formulation comprises glycerin.

Aspect 14. The nebulizer device of Aspect 11, wherein the liquid therapeutic formulation is buffered to a pH of about 8.3 to about 8.7.

Aspect 15. A formulation useful for treating sore throat, throaty cough, xerostomia, pharyngitis, laryngitis, postoperative pain, dysphagia, laryngeal granuloma, vocal optimization, laryngopharyngeal reflux or dysbiosis wherein the formulation comprises an aqueous solution comprising one or more active ingredients selected from the group consisting of marshmallow root, slippery elm extract, aloe vera extract, manuka honey, honey, holy basil, menthol, spearmint, capsaicin, propolis, curcumin, xylitol, turmeric, probiotics, and calendula nebulized into aerosolized particles in a range of 10 to 35 microns.

Aspect 16. The formulation of Aspect 16, comprising marshmallow root, slippery elm extract, aloe vera extract, and manuka honey.

Aspect 17. The formulation of Aspect 15, comprising glycerin.

Aspect 18. The formulation of Aspect 15, wherein the formulation is buffered to a pH of about 8.3 to about 8.7.

Aspect 19. The formulation of Aspect 15, comprising aerosolized particles in a range of 15 to 23 microns.

Aspect 20. A method for treating sore throat, throaty cough, xerostomia, pharyngitis, laryngitis, postoperative pain, dysphagia, laryngeal granuloma, vocal optimization, laryngopharyngeal reflux or dysbiosis in a subject, the method comprising: providing an aqueous solution comprising one or more active ingredients selected from the group consisting of marshmallow root, slippery elm extract, aloe vera extract, manuka honey, honey, holy basil, menthol, spearmint, capsaicin, propolis, curcumin, xylitol, turmeric, probiotics, and calendula; nebulizing the aqueous solution into aerosolized particles in a range of 3 to 35 microns, optionally from about 10 to about 35 microns; and delivering the aerosolized particles to selected areas of the upper airway of the subject.

Aspect 21. The method of Aspect 20, wherein providing the aqueous solution, nebulizing the aqueous solution and delivering the aerosolized particles are carried out using a nebulizer device comprising: a housing comprising a mouthpiece and a cover movable between a first position and a second position; a reservoir inside the housing configured to hold a liquid therapeutic formulation; a vibratory mesh nebulizer inside the housing in fluid communication with the reservoir and the mouthpiece, wherein the nebulizer is configured to nebulize the liquid therapeutic formulation into aerosolized particles in a range of 10 to 35 microns; wherein when the cover is in the first position the cover covers the mouthpiece and inactivates the nebulizer and when the cover is in the second position the mouthpiece is exposed to the subject and the device is activated to nebulize the liquid therapeutic formulation when the mouthpiece of the device is inserted into the oral cavity of the subject.

Aspect 22. A nebulizer device, comprising: a housing comprising a mouthpiece and a cover movable between a first position and a second position, the nebulizer device being configured to contain therein a liquid therapeutic formulation; and a vibratory nebulizer being disposed inside the housing and being in fluid communication with the liquid therapeutic formulation and the mouthpiece, the vibratory nebulizer optionally comprising a mesh, the vibratory nebulizer configured to nebulize the liquid therapeutic formulation into aerosolized particles in a range of 3 to 35 microns, optionally from about 10 to about 35 microns, and the nebulizer device configured such that when the cover is in the first position the cover obstructs the mouthpiece and inactivates the nebulizer and when the cover is in the second position the mouthpiece is exposed to a user.

FIGS. 9A-9B show an example cover 3, which cover can be moved between first and second positions. It should be understood, however, that a cover need not be wedge-shaped in configuration, as a cover can also be, for example, box-shaped or even be a panel, tab, or other element that can at least partially obstruct the mouthpiece. As but one example, a cover can be a panel that slides across or even within the mouthpiece.

Aspect 23. The nebulizer device of Aspect 22, wherein when the cover is in the second position, the nebulizer device is activated to nebulize the liquid therapeutic formulation. Without being bound to any particular theory or embodiment, the nebulizer device can include a switch or other sensor that modulates the operation of the device in response to the position of the cover and/or in response to the position of a switch or other feature that controls the position of the cover. As an example, a cover can be actuated by a button, slider, or other element.

Aspect 24. The nebulizer device of any one of Aspects 22-23, wherein the vibratory nebulizer comprises a mesh, the mesh optionally being a piezoelectric mesh. Without being bound to any particular theory or embodiment, piezoelectric meshes are considered particularly suitable. Such a piezoelectric mesh can include, for example, piezoelectric elements-which can be discs, rings, or other forms—that excite a membrane; such a membrane can be in the form of a mesh or in the form of a membrane having apertures formed therethrough.

Aspect 25. The nebulizer device of any one of Aspects 22-24, wherein the nebulizer device is configured to deliver the aerosolized particles to a subject via passive nebulization.

Aspect 26. The nebulizer device of any one of Aspects 22-24, wherein the nebulizer device is configured to deliver the aerosolized particles to a subject via nebulization at a velocity of less than about 10 m/s, optionally at about 1 m/s. A device according to the present disclosure can include a fan or other element that encourages the movement of aerosolized particles, but this is not a requirement.

Aspect 27. The nebulizer device of any one of Aspects 22-26, wherein the cover rotates about an axis. Such a configuration is shown in FIGS. 9A-9B, which show the movement of rotatable cover 3.

Aspect 28. The nebulizer device of any one of Aspects 22-27, wherein the housing is circular in cross-section. A housing, however, need not be circular in cross-section, as a housing can be polygonal, oblong, or other-shaped in cross-section. An example circular housing is provided in FIG. 9A; as shown, housing 2 is circular in cross-section.

Aspect 29. The nebulizer device of Aspect 28, wherein the housing is characterized as a cylinder having a radius and a height, the radius being greater than the height.

Aspect 30. The nebulizer device of any one of Aspects 22-29, wherein the housing contains therein the liquid therapeutic formulation.

Aspect 31. The nebulizer device of any one of Aspects 22-30, wherein the mouthpiece is removable. The mouthpiece can be removed, for example, for cleaning purposes.

Aspect 32. The nebulizer device of any one of Aspects 22-31, further comprising a funnel, the funnel placing the liquid therapeutic formulation into fluid communication with the vibratory nebulizer. As shown in FIG. 12, funnel 30 can include an opening 31. Opening 31 can mate with an opening in a reservoir (if present), but this is not a requirement. Opening 31 can also be in fluid communication with liquid therapeutic formulation inside the housing when a reservoir is not present.

Aspect 33. The nebulizer device of Aspect 32, wherein the funnel defines a path comprising at least one bend. Such a path can therefore allow a user to communicated therapeutic formulation to the vibratory nebulizer while maintaining a desired form factor for the device.

Aspect 34. The nebulizer device of Aspect 33, wherein the funnel defines a path comprising a 90° bend.

Aspect 35. The nebulizer device of any one of Aspects 22-34, further comprising a reservoir disposed within the housing, the reservoir configured to contain therein the liquid therapeutic formulation. In such devices, the liquid therapeutic formulation can be contained within the reservoir, and the reservoir can in turn be disposed within the housing.

Aspect 36. The nebulizer device of Aspect 35, wherein the reservoir is removable. Such a reservoir can thus be refilled. Such a device can be part of a kit in which different reservoirs can be used in connection with a given device.

Aspect 37. The nebulizer device of any one of Aspects 22-36, further comprising a controller configured to modulate operation of the vibratory nebulizer. It should be understood that the controller can modulate the on/off state of the device, but can also, in some cases, modulate the operation of the vibratory nebulizer. For example, the controller can modulate the operation of the vibratory nebulizer so as to control the droplet size of the aerosolized droplets. This can be accomplished, for example, by preprogramming the controller to give rise to droplets of a particular size.

Aspect 38. The nebulizer device of any one of Aspects 22-37, further comprising a power cell. Such a power cell can be, for example a battery. A device according to the present disclosure can also, however, be configured such that the device receives power from a wired power source, for example from a wall plug.

Aspect 39. The nebulizer device of Aspect 38, wherein the power cell is removable.

Aspect 40. The nebulizer device of Aspect 38, wherein the power cell is rechargeable.

Aspect 41. A method, comprising operating a nebulizer device according to any one of Aspects 22-40 so as to deliver aerosolized particles of a liquid therapeutic formulation to a subject. The subject can, as described herein, inhale the droplets produced by the nebulizer.

Aspect 42. A nebulizer device, comprising: a housing configured to contain therein a liquid therapeutic formulation; a vibratory nebulizer disposed within the housing; and the nebulizer device being convertible between (1) a first state in which fluid communication between a subject and the liquid therapeutic formulation is interrupted and (2) a second state in which fluid communication is effected between the subject and the liquid therapeutic formulation and the nebulizer device is activated to nebulize the liquid therapeutic formulation. Such a device can be handheld so as to be portable. Such a device can, in some embodiments, include an integrated mouthpiece.

Aspect 43. The nebulizer device of Aspect 42, further comprising a reservoir disposed within the housing and configured to contain the liquid therapeutic formulation. In some embodiments, the liquid therapeutic formulation is contained within the housing, without the presence of a separate reservoir.

Aspect 44. The nebulizer device of Aspect 42, wherein the reservoir is removeable.

Aspect 45. The nebulizer device of any one of Aspects 42-44, further comprising a mouthpiece, the mouthpiece configured to place the subject into fluid communication with the vibratory nebulizer.

Aspect 46. The nebulizer device of Aspect 45, further comprising a cover configured to obstruct the mouthpiece. Exemplary covers are described elsewhere herein.

Aspect 47. The nebulizer device of Aspect 46, wherein moving the cover converts the nebulizer device between the first state and the second state.

Aspect 48. The nebulizer device of any one of Aspects 42-47, wherein the vibratory nebulizer is configured to nebulize the liquid therapeutic formulation into aerosolized particles in a range of 3 to 35 microns, optionally from about 10 to about 35 microns.

Aspect 49. A method, comprising determining an effective aerosol particle size and an effective aerosol velocity. Without being bound to any particular theory or embodiment, the effective aerosol particle size and the velocity of the aerosol can influence drug particle deposition in the larynx.

As an example, a patient-specific laryngeal airway model can be constructed from images, such as computed tomography images of a patient. In non-limiting embodiments, computational fluid and particle dynamics modeling can be used to simulate resting inhalation and oral nebulized drug delivery to the larynx under varying aerosol velocities (e.g., about 0-10 m/s) and particle sizes (about 1-30 microns) in each patient's laryngeal airway model.

The disclosed technology can include adjusting the effective aerosol particle size and the effective velocity of aerosols. For example, the effective aerosol particle size and the effective velocity of aerosols can be adjusted based on models, such as models of a given subject and/or a given population of subjects. In non-limiting embodiments, the nebulized aerosol velocities with the highest laryngeal deposition can be 0 m/s (passive nebulization) and 1 m/s, with average deposition of 21.3% (standard deviation=12.1%) and 18.6% (standard deviation=12.4%), respectively.

Without being bound to any particular theory or embodiment, in some instances, the aerosol particle sizes with the highest laryngeal deposition can be between about 8 microns and about 12 microns. In some non-limiting instances, the nebulized aerosol particles released with lower velocities can be more likely to deposit at the larynx.

In certain embodiments, the effective aerosol particle size and the effective velocity of aerosols can be decreased to achieve improved drug deposition in the larynx via oral nebulization.

The description herein illustrates the principles of the disclosed subject matter. Various modifications and alterations to the described embodiments will be apparent to those skilled in the art in view of the teachings herein. It should be noted that the language used herein has been selected for readability rather than to delineate or limit the disclosed subject matter. Accordingly, the disclosure herein is illustrative, but not limiting, of the scope of the disclosed subject matter. Moreover, the principles of the disclosed subject matter can be implemented in various configurations of hardware and/or software, and are not intended to be limited in any way to the specific embodiments presented herein.

What is claimed:

1. A formulation useful for treating sore throat, throaty cough, xerostomia, pharyngitis,
laryngitis, postoperative pain, dysphagia, laryngeal granuloma, vocal optimization,
laryngopharyngeal reflux or dysbiosis, wherein the formulation comprises:
an aqueous solution comprising one or more active ingredients selected from the group consisting of marshmallow root, slippery elm extract, aloe vera extract, manuka honey, honey, holy basil, menthol, spearmint, capsaicin, propolis, curcumin, xylitol, turmeric, probiotics, and calendula,
the aqueous solution being present as aerosolized particles in a range of 10 to 35 microns.
